(19) United States
(12) Patent Application Publication
(10) Pub. No.: US 2012/0179052 A1
(43) Pub. Date: Jul. 12, 2012

(54) Biodegradable Fluid Delivery Device

(75) Inventors: Grant A. Wilhelm, Plymouth, MN (US); David W. Meyer, Jordan, MN (US)

(73) Assignee: COLDER PRODUCTS COMPANY, St. Paul, MN (US)

(21) Appl. No.: 13/347,362

(22) Filed: Jan. 10, 2012

Related U.S. Application Data
(60) Provisional application No. 61/431,296, filed on Jan. 10, 2011.

(51) Int. Cl. A61B 5/021; A61M 5/14; A61F 7/10; A61J 1/14; F16L 37/00; B23P 21/00

(52) U.S. Cl. 600/485; 137/798; 29/525.01; 607/114; 604/408; 604/257

(57) ABSTRACT

An assembly for a fluid transfer system includes: a connector having a proximal end and an opposite distal end connected by an inner passage; and a container having an internal fluid reservoir. The connector is coupled to the internal fluid reservoir of the container to form a series fluid connection between the inner passage and the internal fluid reservoir, and the connector and the container are made of a biodegradable material.

### BACKGROUND

A tube set is an apparatus including one or more components integrated within a circulatory or delivery fluid transfer system. A hose, wrap pad, and connector of a cold water therapy system is one example of a tube set incorporated within a circulatory fluid transfer system. A sterile fluid container, a length of sterile tube, and various clamps and connectors of an intravenous infusion set is one example of a tube set incorporated within a delivery fluid transfer system. Another example is a blood pressure cuff that is used to deliver a fluid (i.e., air) from a source to a cuff that is expanded around a patient's arm.

Such devices can be problematic when respective components are formed using toxic additives. For example, plastics such as softened and/or stabilized polyvinyl chloride (PVC) can be hazardous to human health, as well as the environment. This may preclude disposal via incineration or recycling. Additionally, landfill disposal may require special handling of the toxic components.

### SUMMARY

In one example, an assembly for a fluid transfer system includes: a connector having a proximal end and an opposite distal end connected by an inner passage; and a container having an internal fluid reservoir; wherein the connector is coupled to the internal fluid reservoir of the container to form a series fluid connection between the inner passage and the internal fluid reservoir, and wherein the connector and the container are made of a biodegradable material.

In another example, a tube set assembly includes: a connector having a proximal end and an opposite distal end connected by an inner passage; a flexible tubing having a first end and an opposite second end; and a flexible container having an internal fluid reservoir; wherein the first end of the tubing is connected to the proximal end of the connector and the second end of the tubing is connected to the internal fluid reservoir of the container to form a series fluid connection between the inner passage and the internal fluid reservoir, and wherein each of the connector, tubing, and container is made of a biodegradable material to form the tube set.

In yet another example, a method for forming a tube set includes: providing a container made of a biodegradable material; positioning a tubing on the container, the tubing being made of a biodegradable material; and positioning a connector on the tubing, the connector being made of a biodegradable material, the tubing forming a fluid passage from the container to the connector.

### DESCRIPTION OF THE DRAWINGS

Aspects of the disclosure may be more completely understood in consideration of the following detailed description of various embodiments of the disclosure in connection with the accompanying drawings.

FIG. 1 is a schematic view of a first example fluid delivery system including a biodegradable tube set assembly.

FIG. 2 is a schematic view of a second example fluid delivery system including a biodegradable tube set assembly.

### DETAILED DESCRIPTION

The present disclosure relates to tube set assemblies. More specifically, the present disclosure relates to fluid transfer systems. Although the present disclosure is not so limited, an appreciation of the various aspects of the disclosure will be gained through a discussion of the examples provided below.

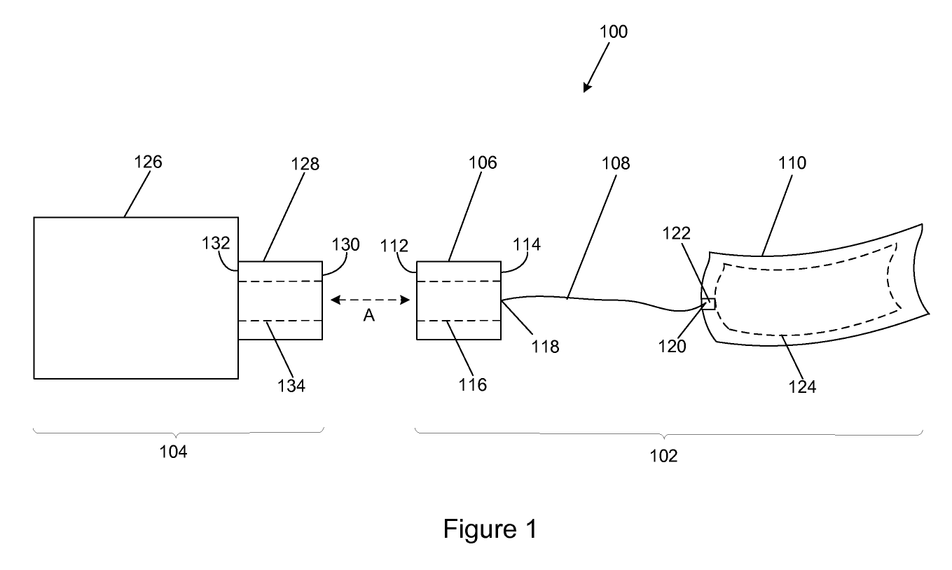

Referring now to FIG. 1, a first example fluid transfer system 100 incorporating aspects of the present disclosure is shown. The fluid transfer system 100 is a single line fluid transfer system and includes a tube set assembly 102 and a fluid control 104.

As used herein, the term “fluid” means any substance that can be made to flow including, but is not limited to, liquids, gases, granular or powdered solids, mixtures or emulsions of two or more fluids, suspensions of solids within liquids or gases, etc.

The example tube set assembly 102 includes a first coupling 106, a conduit 108, and a container 110 coupled together in a series configuration. The first coupling 106 is a connector and includes a distal end 112 and an opposite proximal end 114 connected by an internal channel 116. The conduit 108 is a length of rigid or flexible tubing and includes a first end 118 and an opposite second end 120. The container 110 is a rigid or flexible fluid receptacle and includes an inlet 122 connected to an internal reservoir 124.

The first end 118 of the conduit 108 is connected to the proximal end 114 of the first coupling 106. The second end 120 of the conduit 108 is connected to the inlet 122 of the container 110. In this manner, a series connection is formed between the internal channel 116 of the first coupling 106 and the internal reservoir 124 of the container 110. Other embodiments of the tube set assembly 102 are possible as well.

The example fluid control 104 includes an optional control device 126 and a second coupling 128 integrated with the control device 126. The control device 126 is configured to control transfer of fluid within the fluid transfer system 100. The second coupling 128 is a connector and includes a distal end 130 and an opposite proximal end 132 connected by an internal channel 134, designated in FIG. 1 as an intermittent line. In general, the proximal end 132 of the second coupling 128 is connected to a plurality of internal fluid control features (not shown) of the control device 126. Other embodiments of the fluid control 104 are possible as well.

In general, the first and second couplings 106, 128 may be any type of fitting of a typical coupling assembly. For example, in some embodiments, the first and second couplings 106, 128 are a barbed or Luer-type fitting of a Luer-type coupling assembly. In other embodiments, the first and second couplings 106, 128 are a valved or a non-valved quick connect/disconnect fitting. For example, in some embodiments, the first and second couplings 106, 128 together form a quick connect/disconnect assembly such as that disclosed in U.S. Pat. No. 5,104,158 filed on May 31, 1991, the entirety of which is hereby incorporated by reference. Still other configurations are possible.

In use, the internal channel 116 of the first coupling 106 is aligned with respect to the internal channel 134 of the second coupling 125 along an axis A. Subsequently, the distal end 112 of the first coupling 106 is positioned adjacent to the distal end 130 of the second coupling 128 to secure the first coupling 106 to the second coupling 128, thereby forming a mated coupling assembly. In this manner, a series fluid connection is formed between the control device 126 and the internal reservoir 124 of the container 110.

In certain embodiments, the fluid transfer system 100 is used as a fluid transfer system for a medical application. For example, the fluid control 104 may be a fluid metering device. The container 110 can be, for example, a bladder, a cuff, a hydration pouch, a soap/cleaner solution pouch, a “bag-in-box” pouch, or a pouch used for blood pressure monitoring, cold therapy, intravenous fluid delivery, and other types of medical applications. The container 110 can be rigid or flexible. The example fluid transfer system 100 may be used in a plurality of other applications as well.

In example embodiments, each of the respective elements of the example tube set assembly 102 is formed at least in part from a biodegradable material. For example, in certain embodiments, the first coupling 106 includes biodegradable components (e.g., housing, valve, termination/adaptor, o-rings or other seal types, etc.) formed from rigid/and or flexible biodegradable materials such as Biograde® C 7500 CL from FKuR Kunststoff GmbH of Germany or apinat® from API SpA of Italy.

The first coupling 106 may additionally include metallic components (e.g., bias spring, etc.) formed from rigid/and or flexible metallic materials, such as music wire for example. In the example embodiment, a metal and/or metal alloy is selected according to corrosion/oxidation properties. For example, a specific metal and/or metal alloy may be selected such as to corrode/oxidize more quickly in comparison to other metal materials. In some embodiments, the conduit 108 and container 110 are each respectively formed from a flexible biodegradable thermoplastic elastomer, such as APINAT® from API Spa of Italy.

Other biodegradable materials of the respective elements of the example tube set assembly 102 are possible as well. For example, each of the first coupling 106, the conduit 108, and the container 110 may be selectively formed from various types and percentages of synthetic and/or organic biodegradable and biocompatible materials to achieve desired compostability goals, performance goals, and other goals such as to conform to certain specifications for a given application.

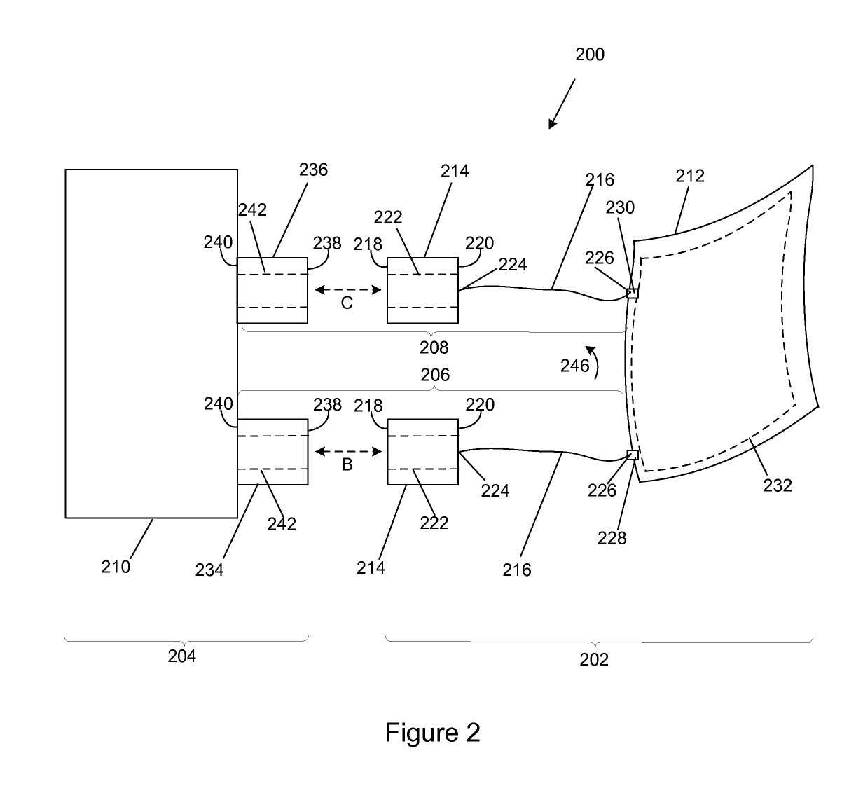

Referring now to FIG. 2, a second example fluid transfer system 200 incorporating aspects of the present disclosure is shown. In general, the second example fluid transfer system 200 is configured similar to the first example fluid transfer system 100 as described above.

The fluid transfer system 200 is a two-line fluid transfer system and includes a tube set assembly 202 and a fluid control 204. The example tube set assembly 202 includes a fluid supply path 206, a fluid return path 208, and a container 212. The fluid supply path 206 and the fluid return path 208 are similarly configured and generally allow for fluid transfer between a control device 210 of the fluid control 204 and the container 212 of the tube set assembly 202. Each of the fluid supply path 206 and the fluid return path 208 include a coupling 214 and a conduit 216 coupled together in a series configuration. The coupling 214 is a connector and includes a distal end 218 and an opposite proximal end 220 connected by an internal channel 222. The conduit 216 is a length of rigid or flexible tubing and includes a first end 224 and an opposite second end 226. The container 212 is a rigid or flexible fluid receptacle and includes an inlet 228 and an outlet 230 each connected to an internal reservoir 232.

The first end 224 of the conduit 216 is connected to the proximal end 220 of the first coupling 214. The second end 226 of the conduit 216 of the fluid supply path 206 is connected to the inlet 228 of the container 212. Similarly, the second end 226 of the conduit 216 of the fluid return path 208 is connected to the outlet 230 of the container 212.

The control device 210 of the example fluid control 204 includes a fluid control supply coupling 234 and a fluid control return coupling 236. The control device 210 is generally configured to control transfer of fluid within the fluid transfer system 200. The fluid control supply coupling 234 and the fluid control return coupling 236 are each a connector and similarly configured including includes a distal end 238 and an opposite proximal end 240 connected by an internal channel 242, designated in FIG. 2 as an intermittent line. In general, the proximal end 240 of the fluid control supply coupling 234 and the fluid control return coupling 236 are each respectively connected to a plurality of corresponding internal fluid control features (not shown) of the control device 210.

Other embodiments of the control device 210 are possible as well. For example, in some embodiments, the fluid control supply coupling 234 and the fluid control return coupling 236 are formed as a single coupling with multiple coaxial fluid flow paths, such as for example, the Twin Tube™ coupling from Colder Products Corporation of St. Paul, Minn. Other multiple line couplings are possible as well.

In use, the internal channel 222 of the coupling 214 of the fluid supply path 206 is aligned with respect to the internal channel 242 of the fluid control supply coupling 234 along an axis B. Subsequently, the distal end 218 of the coupling 214 of the fluid return path 208 is positioned adjacent to the distal end 238 of the fluid control supply coupling 234 to secure the respective coupling 214 to the fluid control supply coupling 234, thereby forming a mated coupling assembly.

Similarly, the internal channel 222 of the coupling 214 of the fluid return path 208 is aligned with respect to the internal channel 242 of the fluid control return coupling 236 along an axis C. Subsequently, the distal end 218 of the coupling 214 of the fluid return path 208 is positioned adjacent to the distal end 238 of the fluid control return coupling 236 to secure the respective coupling 214 to the fluid control return coupling 236, thereby forming a mated coupling assembly. In this manner, a circuitous fluid connection, having fluid flow for example in a counterclockwise direction 246 as shown in FIG. 2, is formed between the control device 210 and the internal reservoir 232 of the container 212.

In general, the fluid control supply coupling 234, fluid control return coupling 236, and coupling 214 may be any type of fitting of a typical coupling assembly, such as described above with respect to the first and second couplings 106, 128 of the fluid transfer system 100. Additionally, each of the respective elements of the example tube set assembly 202 may be selectively formed in part from rigid and/or flexible biodegradable materials and rigid and/or flexible metallic materials of choice, also as described above with respect to the fluid transfer system 100.

In some embodiments, the fluid transfer system 200 is used as a fluid transfer system for a medical application. For example, the fluid control 204 may be a fluid metering device, and the container 212 may be a bladder, a cuff, or a pouch used for blood pressure management, cold therapy, intravenous fluid delivery, and other types of medical applications. However, the example fluid transfer system 200 may be used in a plurality of other applications as well.

The various embodiments described above are provided by way of illustration only and should not be construed to limiting. Various modifications and changes that may be made to the embodiments described above without departing from the true spirit and scope of the disclosure.

What is claimed is:

1. An assembly for a fluid transfer system, comprising:
a connector having a proximal end and an opposite distal end connected by an inner passage; and
a container having an internal fluid reservoir;
wherein the connector is coupled to the internal fluid reservoir of the container to form a series fluid connection between the inner passage and the internal fluid reservoir, and
wherein the connector and the container are made of a biodegradable material.

2. The assembly of claim 1, further comprising a control device configured to control transfer of fluid within the fluid transfer system.

3. The assembly of claim 1, wherein the connector is a barbed or a Luer-type fitting.

4. The assembly of claim 1, wherein the connector is a valved or a non-valved quick connect/disconnect fitting.

5. The assembly of claim 1, further comprising a tubing having a first end and a second end, wherein the first end of the tubing is connected to the proximal end of the connector and the second end of the tubing is connected to the internal fluid reservoir of the container to form the series fluid connection between the inner passage and the internal fluid reservoir, wherein the tubing is made of a biodegradable material.

6. The assembly of claim 5, wherein the tubing is flexible.

7. The assembly of claim 1, wherein the container is a bladder, a cuff, a hydration pouch, cleaner solution pouch, or a bag-in-box pouch.

8. The assembly of claim 1, wherein the container is a pouch used for blood pressure monitoring, cold therapy, or intravenous fluid delivery.

9. The assembly of claim 1, wherein the container is flexible.

10. A tube set assembly, comprising;
a connector having a proximal end and an opposite distal end connected by an inner passage;
a flexible tubing having a first end and a second end; and
a flexible container having an internal fluid reservoir;
wherein the first end of the tubing is connected to the proximal end of the connector and the second end of the tubing is connected to the internal fluid reservoir of the container to form a series fluid connection between the inner passage and the internal fluid reservoir, and
wherein each of the connector, tubing, and container is made of a biodegradable material to form the tube set assembly.

11. The tube set assembly of claim 10, further comprising a control device.

12. The tube set assembly of claim 10, wherein the connector is a barbed or a Luer-type fitting.

13. The tube set assembly of claim 10, wherein the connector is a valved or a non-valved quick connect/disconnect fitting.

14. The tube set assembly of claim 10, wherein the container is a bladder, a cuff, a hydration pouch, cleaner solution pouch, or a bag-in-box pouch.

15. The tube set assembly of claim 10, wherein the container is a pouch used for blood pressure monitoring, cold therapy, or intravenous fluid delivery.

16. A method for forming a tube set, the method comprising:
providing a container made of a biodegradable material;
positioning a tubing on the container, the tubing being made of a biodegradable material; and
positioning a connector on the tubing, the connector being made of a biodegradable material, the tubing forming a fluid passage from the container to the connector.

17. The method of claim 16, further comprising allowing a control device to control a transfer of fluid flowing through the tube set.

18. The method of claim 16, wherein the connector is a barbed or a Luer-type fitting.

19. The method of claim 16, wherein the container is a bladder, a cuff, a hydration pouch, cleaner solution pouch, or a bag-in-box pouch.

20. The method of claim 16, wherein the container is a pouch used for blood pressure monitoring, cold therapy, or intravenous fluid delivery.
